(19) United States
(12) Patent Application Publication
(10) Pub. No.: US 2023/0013382 A1
(43) Pub. Date: Jan. 19, 2023

(54) Surgical Spoons for Veterinary Use

(71) Applicant: BOARD OF TRUSTEES OF THE UNIVERSITY OF ARKANSAS, Little Rock, AR (US)

(72) Inventors: Davina Cistelle D'Angelo, Fayetteville, AR (US); Lauren Thomas, Fayetteville, AR (US); Morten Olgaard Jensen, Fayetteville, AR (US); Sam Stephens, Fayetteville, AR (US)

(73) Assignee: BOARD OF TRUSTEES OF THE UNIVERSITY OF ARKANSAS, Little Rock, AR (US)

(21) Appl. No.: 17/812,687

(22) Filed: Jul. 14, 2022

Related U.S. Application Data
(60) Provisional application No. 63/221,893, filed on Jul. 14, 2021.

(51) Int. Cl. A61B 17/22

(52) U.S. Cl. CPC A61B 17/22031; A61M 2210/1085; A61B 2017/22034

(57) ABSTRACT

A surgical spoon for removing uroliths having a head connected to a handle by a neck, The head has a perimeter having a curved edge and at least one straight edge.

### RELATED APPLICATIONS

This application claims priority to U.S. Provisional Application No. 63/221,893, filed on Jul. 14, 2021, which is incorporated herein in its entirety.

### STATEMENT REGARDING FEDERALLY SPONSORED RESEARCH & DEVELOPMENT

Not applicable.

### INCORPORATION BY REFERENCE OF MATERIAL SUBMITTED ON A COMPACT DISC

Not applicable.

### BACKGROUND OF THE INVENTION

Bladder stones, also known as uroliths, are not an uncommon condition in domestic dogs. To remove these uroliths, which occur in various sizes, shapes, and compositions, veterinarians often perform a surgical procedure called a cystotomy. A cystotomy consists of creating a small incision in the urinary bladder and the utilization of various methods to extract the uroliths. Patients in need of a cystotomy experience difficulty urinating (stranguria), blood in the urine (hematuria), discomfort, urinary tract infections, and even urinary blockage and obstruction in more severe cases. For general veterinary practitioners, the laparoscopic-assisted technique is unavailable. As a result, the methods available for stone extraction are often limited to flushing the stones out by inserting a urinary catheter through the urinary tract and the use of off-label devices such as the standard tablespoon, teaspoon, or gallbladder spoon. Many times, these methods still make it difficult to remove all of the stones, especially smaller ones that can be millimeters in size. Remaining uroliths can cause post-operative complications and discomfort to the patient. Additionally, the tissue of the urinary bladder contracts when an incision is made, creating an additional challenge of maneuvering effectively within the lumen of the urinary bladder when off-label devices are implemented. Failure to remove all uroliths is a breach in veterinary care because the uroliths can cause post-operative complications and can leave an animal in urinary discomfort. In fact, studies have reported that uroliths were left behind in 20% of dogs after cystotomy (Langston, Gisselman, Palma, & McCue, 2010). It is important that all uroliths are removed because recurrence is very common. Small uroliths can cause lower urinary tract obstruction if left untreated (Appel, Otto, & Weese, 2012). Urinary tract infections can occur but are not as common; however, it is still important to prevent (Pinel, Monnet, & Reems, 2013). Some veterinarians will also administer anti-inflammatory therapy to the patient as well (Franz et. al, 2009). The composition of some uroliths can make recurrence more likely, such as calcium oxalate stones (Bartges, et. al, 1999). The typical cystotomy surgical time for canines, in hours, is 1.26±0.50 (Arulpragasam, S. P., Case, J. B., & Ellison, G. W., 2013). The difficulty in extracting smaller uroliths increases the period of time anesthesia is administered to the patient and cost to the client. In fact, prolonged anesthesia can cause an animal harm (Gaynor, et. al, 1999), and potentially “may cause a significant delay in gastric emptying and predispose to post-anesthetic GI complications” (Boscan, P., Cochran, S., Monnet, et al., 2014). By decreasing surgical time, the veterinarian will also benefit from more allotted time for other surgeries and tasks of the day, which can have a positive impact on the veterinary practice as a whole.

### BRIEF SUMMARY OF THE INVENTION

In one embodiment, the present invention concerns surgical spoons which differ in size and shape. The medical spoons are targeted for use in the veterinary industry, specifically to be used during the cystotomy surgery in order to remove all uroliths more easily from a patient's urinary bladder.

In other embodiments, the present invention concerns surgical spoons for cystotomy surgery on an animal such as a canine, feline and exotics.

In other embodiments, the present invention concerns surgical spoons designed with straight edges in order to maneuver the instrument more effectively inside the urinary bladder.

In other embodiments, the present invention concerns surgical spoons designed with straight edges wherein the edges allow more surface area contact between the spoon head and urinary bladder wall to facilitate the efficient and complete removal of the uroliths.

In other embodiments, the present invention concerns surgical spoons wherein straight edges of the surgical spoons have slightly rounded corners which decrease the sharpness and prevent damage to the tissue of the urinary bladder while gently scooping out uroliths.

### BRIEF DESCRIPTION OF THE SEVERAL VIEWS OF THE DRAWINGS

In the drawings, which are not necessarily drawn to scale, like numerals may describe substantially similar components throughout the several views. Like numerals having different letter suffixes may represent different instances of substantially similar components. The drawings illustrate generally, by way of example, but not by way of limitation, a detailed description of certain embodiments discussed in the present document.

FIG. 1 is a top view of a first embodiment of the present invention.

FIG. 2 is a side view of the embodiment shown in FIG. 1.

FIG. 3 is a front view of the embodiment shown in FIG. 1.

FIG. 4 is a top view of a second embodiment of the present invention.

FIG. 5 is a side view of the embodiment shown in FIG. 4.

FIG. 6 is a front view of the embodiment shown in FIG. 4.

FIG. 7 is a top view of a third embodiment of the present invention.

FIG. 8 is a side view of the embodiment shown in FIG. 7.

FIG. 9 is a front view of the embodiment shown in FIG. 7.

FIG. 10 is a top view of a fourth embodiment of the present invention.

FIG. 11 is a side view of the embodiment shown in FIG. 10.

FIG. 12 is a top view of a fifth embodiment of the present invention.

FIG. 13 illustrates the embodiment shown in FIGS. 10-12 inside a bladder.

FIG. 14 illustrates the embodiment shown in FIGS. 10-12 inside a bladder from a side perspective.

FIG. 15 illustrates the embodiment shown in FIGS. 10-12 inside a bladder from a top perspective.

### DETAILED DESCRIPTION OF THE INVENTION

Detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a representative basis for teaching one skilled in the art to variously employ the present invention in virtually any appropriately detailed method, structure, or system. Further, the terms and phrases used herein are not intended to be limiting, but rather to provide an understandable description of the invention.

The embodiments of the present invention concern surgical spoons for cystotomy surgeries in patients such as canines. The surgical spoons are not limited by the size, shape, breed of the animal. The spoons differ from traditional tablespoons by the placement of the straight edges on the head of the instrument. The edges increase the surface area available to gently scrape and scoop out various sized uroliths. The surgical spoon sizes are designed to fit inside a urinary bladder that has contracted during surgery.

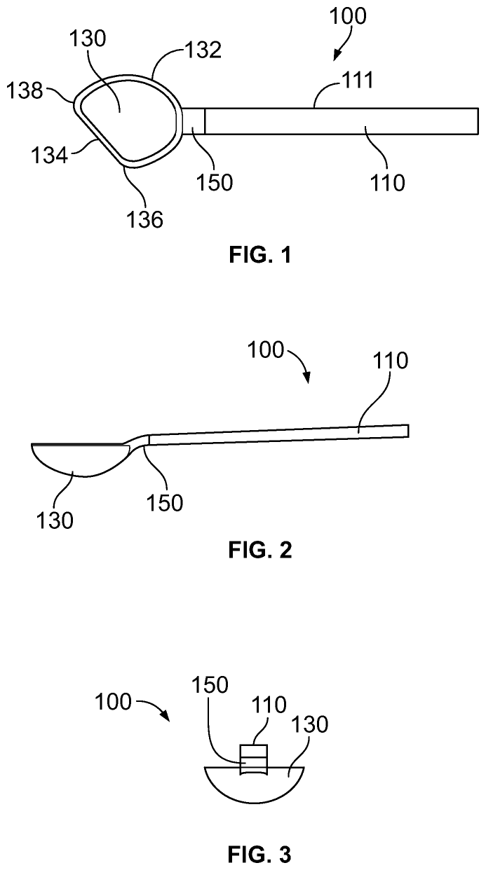

FIGS. 1-3 illustrate a first embodiment of the present invention. As shown spoon 100 includes handle 110 to which head 130 is attached by neck 150. The perimeter of head 130 consists of curved edge 132 which is truncated by straight edge 134. Rounded corners 136 and 138 connect edges 132 and 134. The length of head 130 as measured from where head 130 connects to neck 150 to rounded corner 138 is 0.97 inches. The width of head 130 as measured from corner 136 to the furthest point on edge 132 is 0.90 inches.

Lastly, straight edge 134 is angled rearwardly with respect to the long axis 111 of handle 110. This creates a tapered appearance for head 130 with corner 138 serving as the apex of the taper or the tip of the device.

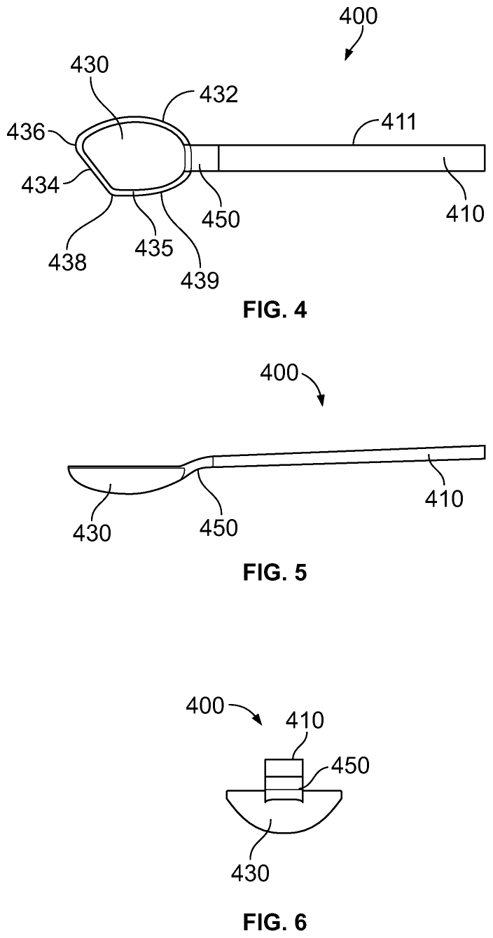

FIGS. 4-6 illustrate a second embodiment of the present invention. As shown spoon 400 includes handle 410 to which head 430 is attached by neck 450. As shown in FIG. 5, neck 450 may be curved. The perimeter of head 430 consists of curved edge 432 which is truncated by straight edges 434 and 435.

Rounded corner 436 connects straight edges 434 to curved edge 432. and 134. Rounded corner 438 connects straight edge 434 to straight edge 435. Rounded corner 439 connects edge 435 to edge 432.

The length of head 430 as measured from where head 430 connects to neck 450 to rounded corner 438 is 1.07 inches. The width of head 430 as measured from edge 435 to the furthest point on edge 432 is 0.78 inches.

Lastly, straight edge 434 is angled rearwardly with respect to the long axis 411 of handle 410. This creates a tapered appearance for head 430 with corner 436 serving as the apex of the taper or the tip of the device.

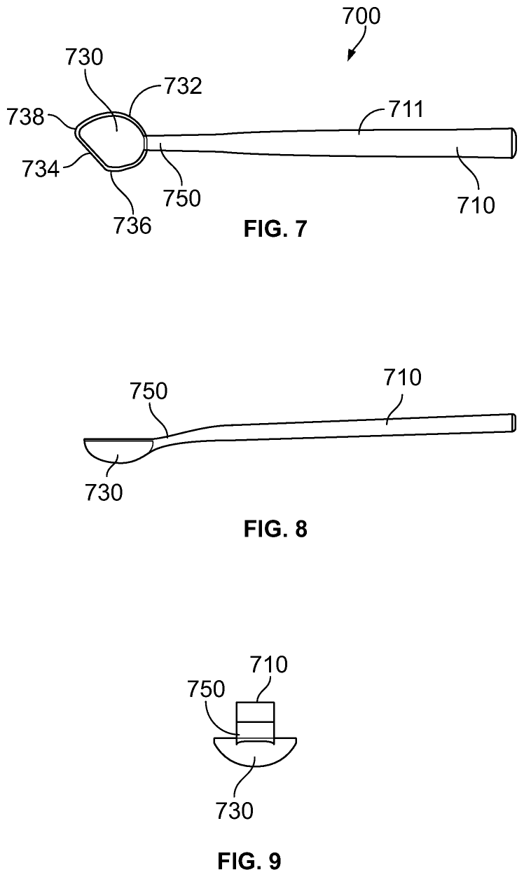

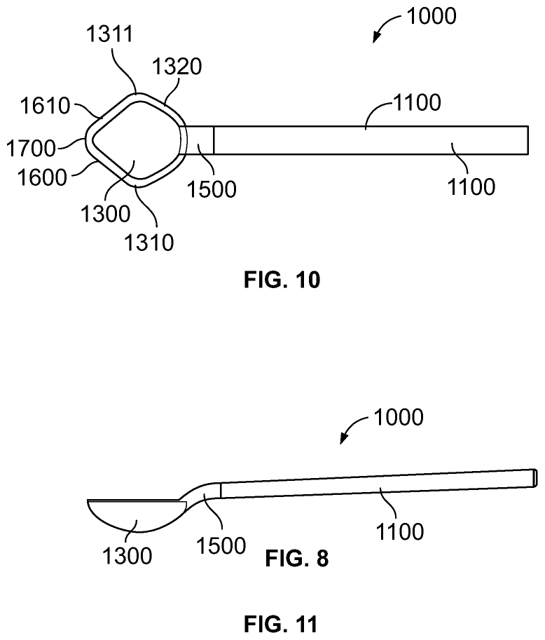

FIGS. 7-9 illustrate a third embodiment of the present invention. As shown spoon 700 includes handle 710 to which head 730 is attached by neck 750. The perimeter of head 730 consists of curved edge 732 which is truncated by straight edge 734. Rounded corners 736 and 738 connect edges 732 and 734. The length of head 730 as measured from where head 730 connects to neck 750 to rounded corner 738 is 0.54 inches. The width of head 730 as measured from corner 736 to the furthest point on edge 732 is 0.490 inches.

Lastly, straight edge 734 is angled rearwardly with respect to the long axis 711 of handle 710. This creates a tapered appearance for head 730 with corner 738 serving as the apex of the taper or the tip of the device.

FIGS. 10 and 11 illustrate a fourth embodiment of the present invention. As shown spoon 1000 includes handle 1100 to which head 1300 is attached by neck 1500. As shown in FIG. 11, neck 1500 may be curved instead of straight.

The perimeter of head 1300 is conical in shape and formed by curved edge 1320 which is connected to handle 1100. At rounded corners 1310 and 1311, curved edge transitions to opposingly located straight edges 1600 and 1610 which are angled rearwardly with respect to long axis 1110 of handle 1100.

Straight edges 1600 and 1610 terminate at rounded tip 1700 and form an acute angle. In a preferred embodiment, straight edges 1600 and 1610 form an 80 degree angle.

The length of head 1300 as measured from where head 1300 connects to neck 1500 to tip 1700 is 0.80 inches. The width of head 1300 as measured from corner 1310 to 1310 is 0.78 inches. In other embodiments, the width of head 1300 may be 2 inches, 1.75 inches, 1.5 inches or 1.35 inches.

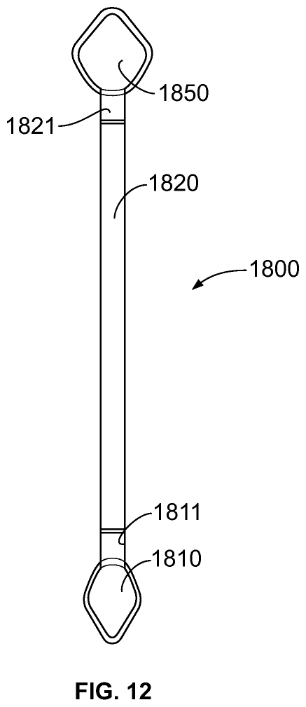

FIG. 12 shows another embodiment of the present invention concerning a two sided surgical spoon 1800. As shown, the device comprises first head 1810 connected to handle 1820 by neck 1811. Also provided is opposingly located second head 1850 which is connected to the opposite end of handle 1820 by neck 1821. The configuration of heads 1810, 1850 as well as necks 1811 and 1821 may be the same as described above.

In a preferred embodiment, first head 1810 is different in shape or configuration than second head 1850. Preferably, one of the heads is smaller in size than the other such as in width, length, or both. Providing different configurations affords additional options for retrieving uroliths to the user when performing a surgical procedure.

When a urinary bladder is empty, it is small in size and requires an instrument that can maneuver gently within the lumen of the urinary bladder tissue. The novel surgical spoons were sketched and designed on a Computer-Aided Design program called SolidWorks. The material of the 3D printed surgical spoons is a Dental Surgical Guide Resin. The resin is Class I Biocompatible, withstands the heat and pressure of autoclave sterilization, and is often used by dental professionals in the operating room and can remain in the mouth for up to 24 hours. The biocompatibility confirms there are no adverse effects in relation to cytotoxicity, sensitivity, irritation, acute toxicity, and genotoxicity. In order to ensure biocompatibility, all FormLabs instructions for printing and post-processing were followed. After printing was complete, the surgical spoons were bathed in a Form Wash, which cleans the finished prints in an agitated bath of 99% isopropyl alcohol, for 10-20 minutes to remove any excess uncured resin. The surgical spoons dried for 30 minutes and were post-cured by exposure to UV light and heat for 30 minutes at 60 degrees Celsius in a Form Cure. The spoons were then manually removed from the support bridges and polished carefully (FormLabs, Inc., 2020).

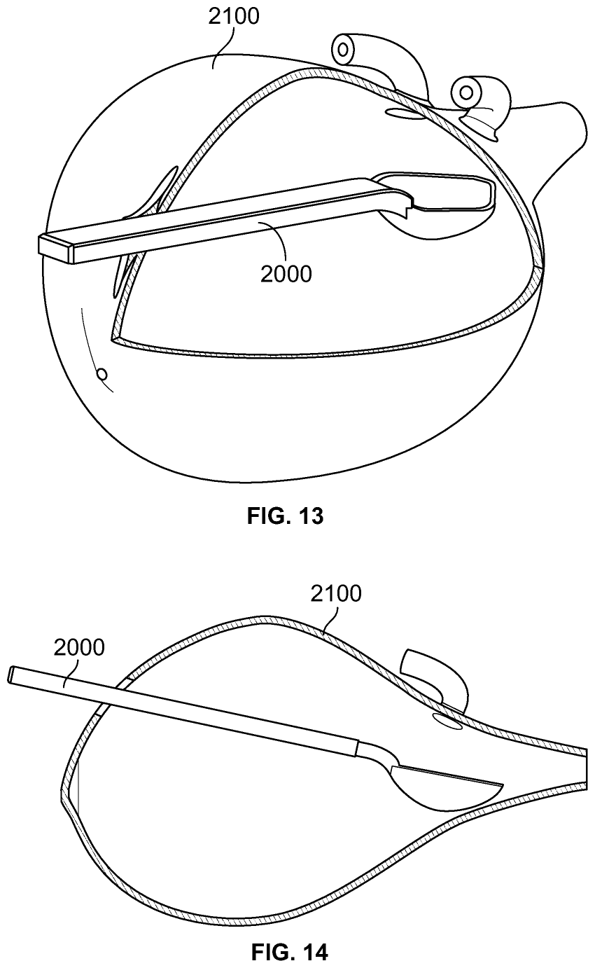

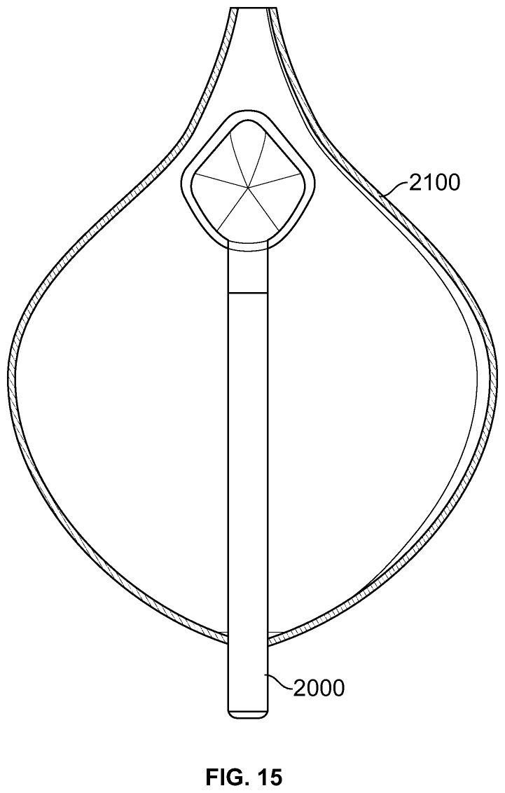

FIGS. 13-15 illustrate how the present invention fits within urinary bladder 2100. As shown, the tapered-shape of the head of spoon 2000 allows access to narrow sections to retrieve objects such as uroliths. As also shown, the straight edges allow for retrieval of objects such as uroliths in the wider sections of the lumen.

While the foregoing written description enables one of ordinary skill to make and use what is considered presently to be the best mode thereof, those of ordinary skill will understand and appreciate the existence of variations, combinations, and equivalents of the specific embodiment, method, and examples herein. The disclosure should therefore not be limited by the above-described embodiments, methods, and examples, but by all embodiments and methods within the scope and spirit of the disclosure.

What is claimed is:

1. A surgical spoon for removing uroliths comprising: a head connected to a handle by a neck; said head having a perimeter having a curved edge and a straight edge; and a curved tip connecting said curved edge to said straight edge.

2. The surgical spoon of claim 1 wherein said straight edge increases the surface area contact between said spoon head and a urinary bladder wall to facilitate a more efficient and complete removal of the uroliths.

3. The surgical spoon of claim 1 wherein said spoon is made from a material having no adverse effects in relation to cytotoxicity, sensitivity, irritation, acute toxicity, and genotoxicity.

4. The surgical spoon of claim 1 wherein said spoon is made from dental surgical resin.

5. A surgical spoon for removing uroliths comprising: a head connected to a handle by a neck; said head having a perimeter having a curved edge and a plurality of straight edges; and a curved tip connecting said curved edge to said straight edge.

6. The surgical spoon of claim 5 wherein said straight edge increases the surface area contact between said spoon head and a urinary bladder wall to facilitate a more efficient and complete removal of the uroliths.

7. The surgical spoon of claim 5 wherein said spoon is made from a material having no adverse effects in relation to cytotoxicity, sensitivity, irritation, acute toxicity, and genotoxicity.

8. The surgical spoon of claim 5 wherein said spoon is made from dental surgical resin.

9. A surgical spoon for removing uroliths comprising: a head connected to a handle by a neck; said head having a perimeter having a curved edge and a first and second straight edge; and a curved tip connecting said first and second straight edges together at an angle.

10. The surgical spoon of claim 9 wherein said straight edges increase the surface area contact between said spoon head and a urinary bladder wall to facilitate a more efficient and complete removal of the uroliths.

11. The surgical spoon of claim 9 wherein said spoon is made from a material having no adverse effects in relation to cytotoxicity, sensitivity, irritation, acute toxicity, and genotoxicity.

12. The surgical spoon of claim 9 wherein said spoon is made from dental surgical resin.

13. The surgical spoon of claim 9 wherein said angle is an acute angle.

14. The surgical spoon of claim 9 wherein said angle is 80 degrees.

15. A surgical spoon for removing uroliths comprising: a first head connected to a handle by a neck; an opposingly located second head connected to said handle by a neck; said first and second heads having a perimeter having a curved edge and a first and second straight edge; and a curved tip connecting said first and second straight edges together at an angle.

16. The surgical spoon of claim 15 wherein said first head and said second head have different configurations.

17. The surgical spoon of claim 16 wherein said first head is smaller than said second head.

18. The surgical spoon of claim 16 wherein said first head has a width that is smaller than the width of said second head.

19. The surgical spoon of claim 15 wherein said angle is an acute angle.

20. The surgical spoon of claim 15 wherein said angle is 80 degrees.
